(19) United States
(12) Patent Application Publication
(10) Pub. No.: US 2024/0206768 A1
(43) Pub. Date: Jun. 27, 2024

(54) METHODS AND SYSTEMS FOR THE ACQUISITION OF KINEMATIC DATA FOR NEUROMOTOR ASSESSMENT

(71) Applicants: POLYVALOR, LIMITED PARTNERSHIP, Montréal (CA); UNIVERSIDAD DE LAS PALMAS DE GRAN CANARIA, Las Palmas de Gran Canaria (ES)

(72) Inventors: Maria Cristina CARMONA DUARTE, Las Palmas de Gran Canaria (ES); Réjean PLAMONDON, Sainte-Julie (CA); Nadir FACI, Longueuil (CA)

(21) Appl. No.: 17/768,623

(22) PCT Filed: Oct. 14, 2020
(86) PCT No.: PCT/CA2020/051372

§ 371 Date: Apr. 13, 2022

Related U.S. Application Data
(60) Provisional application No. 62/916,325, filed on Oct. 17, 2019.

(51) Int. Cl. A61B 5/11; A61B 5/00; G16H 40/67

(52) U.S. Cl. CPC A61B 5/1124; A61B 5/4082; A61B 5/4088; A61B 5/4803; A61B 5/6897; A61B 5/74; G16H 40/67

(57) ABSTRACT

Methods and systems for obtaining kinematic data from a subject for neuromotor assessment are presented herein. The method comprises presenting on a mobile computing device at least two of: a handwriting task, a speech task, and a natural movement task, each task executable by the subject with the mobile computing device, providing an external stimulus through the mobile computing device as a trigger to begin each task and acquiring kinematic data from the subject on the mobile computing device as the tasks are being performed. The acquired kinematic data may be stored locally, processed locally, and/or stored and transmitted remotely for processing.

### CROSS-REFERENCE TO RELATED APPLICATIONS

The present application claims the benefit of U.S. Provisional Patent Application No. 62/916,325 filed on Oct. 17, 2019, the contents of which are hereby incorporated by reference in their entirety.

### TECHNICAL FIELD

The present disclosure relates generally to neuromotor assessments in humans, and more particularly to systems and methods for acquiring kinematic data for neuromotor assessments.

### BACKGROUND OF THE ART

Human movement can be very complex, and there are many past and ongoing studies in various areas of medicine and human sciences dealing with some of these topics. For example in neurosciences, handwriting strokes constitute a specific class of rapid human movements and they are used to study neurodegenerative processes involved in diseases such as Parkinson's and Alzheimer's. The early detection of cerebral lesions appears possible by determining slight deviations from the norm, which are not evident by simple visual inspection.

For the purposes of detection, diagnosis, treatment, and research, various tools have been devised for gathering kinematic data from human subjects. Certain challenges arise when using comparative results from one experiment to another.

Therefore, improvements are needed.

### SUMMARY

In accordance with a broad aspect, there is provided a method for obtaining kinematic data from a subject. The method comprises presenting on a mobile computing device at least two tasks from: a handwriting task, a speech task, and a natural movement task, the at least two tasks executable by the subject with the mobile computing device, providing an external stimulus through the mobile computing device as a trigger to begin the at least two tasks, acquiring kinematic data from the subject on the mobile computing device as the at least two tasks are being performed, and generating a file with the kinematic data together with subject data.

In various embodiments, the method further comprises processing the kinematic data to get parameters that characterize a neuromotor performance of the subject.

In various embodiments, processing the kinematic data comprises processing the kinematic data on the mobile computing device.

In various embodiments, processing the kinematic data comprises decomposing complex movements into simple movements, analyzing statically the simple movements, and characterizing the neuromotor performance of the subject based on the analyzing.

In various embodiments, processing the kinematic data comprises comparing and correlating data from different neuromotor groups acquired through the at least two tasks.

In various embodiments, providing the external stimulus comprises providing a same external stimulus for the at least two tasks.

In various embodiments, providing the external stimulus comprises signaling an end to the at least two tasks.

In various embodiments, the method further comprises transmitting the file with the kinematic data to a remote location.

In various embodiments, the subject data comprises a hardware identifier for the mobile computing device.

In various embodiments, the tasks are presented on the mobile device in accordance with at least one of a specific order and a complexity level.

In accordance with another broad aspect, there is provided a system for obtaining kinematic data from a subject. The system comprises a processing unit and a non-transitory computer readable medium having stored thereon program code. The program code is executable by the processing unit for presenting on a mobile computing device at least two tasks from: a handwriting task, a speech task, and a natural movement task, the at least two tasks executable by the subject with the mobile computing device, providing an external stimulus through the mobile computing device as a trigger to begin the at least two tasks, acquiring kinematic data from the subject on the mobile computing device as the at least two tasks are being performed, and generating a file with the kinematic data together with subject data.

In various embodiments, the program code is further executable for processing the kinematic data to get parameters that characterize a neuromotor performance of the subject.

In various embodiments, processing the kinematic data comprises processing the kinematic data on the mobile computing device.

In various embodiments, processing the kinematic data comprises decomposing complex movements into simple movements, analyzing statically the simple movements, and characterizing the neuromotor performance of the subject based on the analyzing.

In various embodiments, processing the kinematic data comprises comparing and correlating data from different neuromotor groups acquired through the at least two tasks.

In various embodiments, providing the external stimulus comprises providing a same external stimulus for the at least two tasks.

In various embodiments, providing the external stimulus comprises signaling an end to the at least two tasks.

In various embodiments, the program code is further executable transmitting the file with the kinematic data to a remote location.

In various embodiments, the subject data comprises a hardware identifier for the mobile computing device.

In various embodiments, the tasks are presented on the mobile device in accordance with at least one of a specific order and a complexity level.

Features of the systems, devices, and methods described herein may be used in various combinations, in accordance with the embodiments described herein.

### BRIEF DESCRIPTION OF THE DRAWINGS

Reference is now made to the accompanying figures in which:

FIG. 1 is a schematic diagram illustrating an example system for acquiring kinematic data;

FIG. 2 is a schematic diagram illustrating an example handwriting task presented on the system of FIG. 1;

FIG. 3 is a block diagram of an example embodiment for the controller of the system of FIG. 1;

FIG. 4 is a schematic diagram of a network of systems from FIG. 1; and

FIG. 5 is a flowchart of an example method for acquiring kinematic data.

It will be noted that throughout the appended drawings, like features are identified by like reference numerals.

### DETAILED DESCRIPTION

The present disclosure relates to computer-aided neuromotor assessment. Kinematic data is obtained from a subject in order to perform a neuromotor assessment. A mobile computing device is used to acquire the kinematic data from a plurality of muscle groups in order to get parameters that allow for various types of neuromotor assessments. The neuromotor assessment may relate to the diagnosis and/or evolution of a neuromotor disease or upper limb injuries, such as but not limited to Parkinson's disease or Alzheimer's disease, as well as muscular problems, concussions, etc. The neuromotor assessment may relate to evaluating the effect of a treatment for a neuromotor disease or accident. Generally, changes to the health status of a subject may be detected and/or monitored via the motor or neural assessment. Neuromotor assessments combine both motor and neural assessments using the same device and protocol. The neuromotor assessment may also be performed on athletes to track performance and/or practicing effects and/or fatigue.

A single mobile device is used to obtain kinematic data from different muscle groups and their central controller in the motor cortex using two or more modalities. The different modalities on a same device allow comparisons and correlations to be made between the kinematic data from the different neuromotor groups. The single device provides a controlled environment for the acquisition of the kinematic data using different modalities, thus providing a form of synchronization of the kinematic data, and simplifying comparison of the results due to a common reference frame in the acquisition process.

The single mobile device comprises a controller having software configured for presenting one or more tasks to a subject, for each of the modalities available on the mobile device. The combined nature of the modalities allows for coordination in the way the various tasks are presented to the subject, with regards to ordering and/or complexity level (for example of increasing/decreasing complexity). Moreover, the controller may be configured using a global approach to evaluating the subject from multiple perspectives.

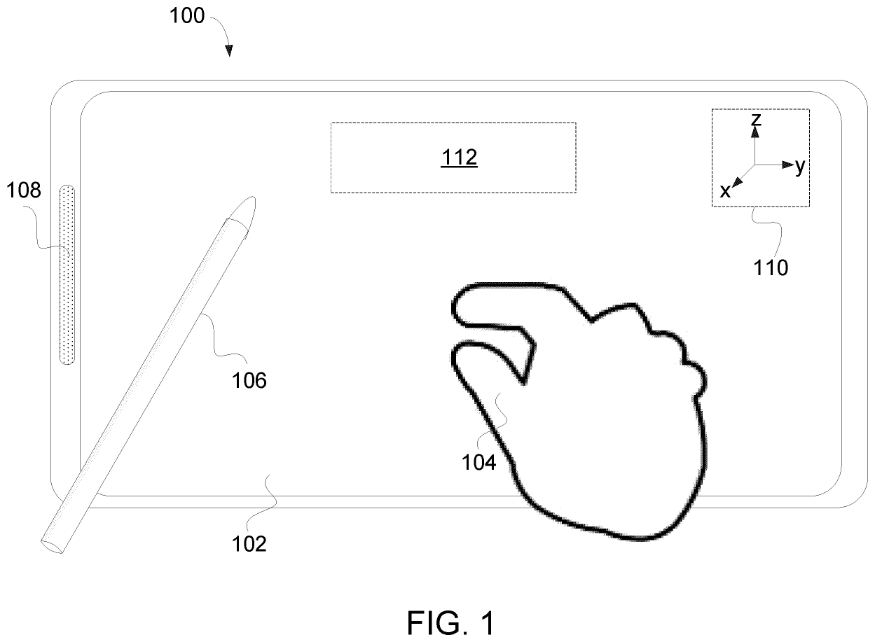

FIG. 1 illustrates an example embodiment for a mobile computing device 100 configured for obtaining kinematic data of muscle groups of a subject using two or more modalities. The mobile computing device 100 may be a tablet computer, such as a slate, a mini tablet, a phablet, a booklet, and Pad and the like. In some embodiments, the mobile computing device 100 is a 2-in-1 portable computer having a detachable or convertible touchscreen. In some embodiments, the mobile computing device 100 is a smartphone. In some embodiments, the mobile computing device 100 is a custom-made device.

A first modality for acquiring kinematic data relates to obtaining a handwriting sample from a subject. Indeed, the generation of handwriting is a complex neuromotor skill requiring the interaction of many cognitive processes. This modality is realized through a touchscreen display 102 of the device 100. The touchscreen display 102 is operated by gestures executed by a hand 104 and/or a digital stylus 106 in contact with the touchscreen display 102. The generated trajectory of the tip of the stylus 106 or a finger is made up of strokes superimposed over time, which may be used as a mathematical description for the impulse response of a neuromuscular system. Kinematic data is acquired by presenting at least one task related to handwriting on the touchscreen display 102. At least one handwriting sample is acquired from the touchscreen display 102 by the device 100 during the handwriting task.

In some embodiments, the touchscreen display 102 covers a portion of a top surface of the device 100, and the other portion may be used for other purposes. In some embodiments, the touchscreen display 102 is separated into two separate regions, a first region dedicated to acquiring handwriting samples from the digital stylus 106 and a second region dedicated to acquiring handwriting samples from the finger of the hand 104. Other embodiments for the touchscreen display 102 may apply depending on practical implementations. For example, a specific area of the display 102 may be dedicated to generated visual stimuli.

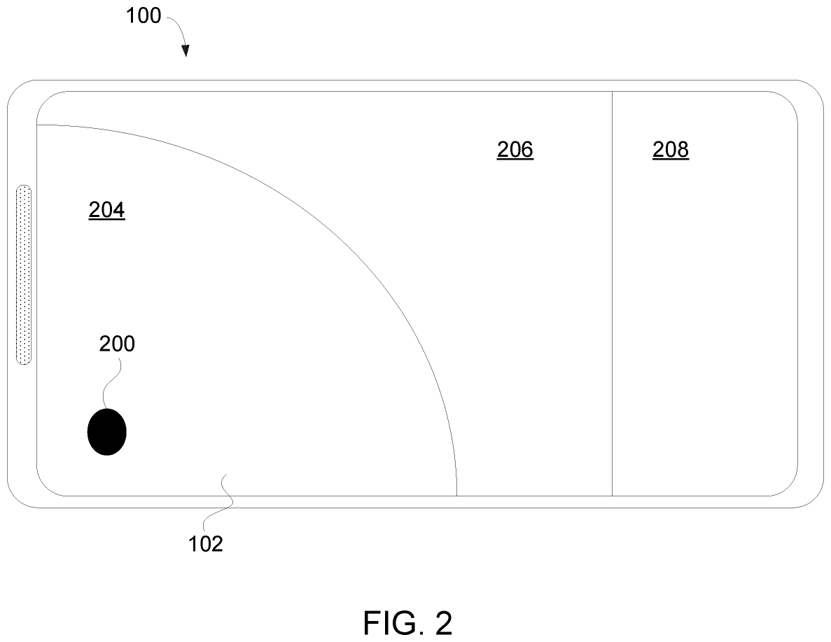

An example task for acquiring a handwriting sample is illustrated in FIG. 2. A starting point, such as a dot 200 is identified. The subject may be asked to draw one or more lines, as quickly as possible, from the starting point 200 to either of the zones 206, 208 outside of the circle 204. Alternatively or in combination therewith, the subject may be asked to draw a shape of a given dimension (i.e. a triangle, a spiral), to move the digital stylus 106 or hand 104 from side to side for a given duration of time (i.e. in an oscillation pattern), to draw a line or a shape with the left hand and/or with the right hand, to write certain letters (separately or joined together), to write a signature, and the like. A wide variety of handwriting tasks may be used to obtain kinematic data suitable for observing, characterizing and evaluating neuromotor control through handwriting patterns.

Visual and/or audio stimuli may be used to indicate the beginning (and in some cases the end) of the handwriting task. For example, a sound cue may be a 1 kHz beep having a duration of 500 ms, or a visual cue may be a colored light that appears on the touchscreen display 102 or on another part of the device 100. A sampling frequency of 200 Hz or greater may be used to acquire the handwriting sample. It can also be smaller, such as 60 Hz, 100 HZ, or any other suitable frequency. The values presented herein are exemplary only and may vary depending on practical implementation.

Referring back to FIG. 1, a second modality for acquiring kinematic data relates to obtaining a speech sample from the subject. This modality is realized through a microphone 108 of the device 100. The microphone 108 converts sound into an electrical signal and may be implemented using any known or other microphone technology, such as a dynamic microphone (also called moving-coil microphone), a condenser microphone (also called capacitor microphone or electrostatic microphone), a piezoelectric microphone, a fiber-optic microphone, a laser microphone, a MEMS (microelectrical-mechanical system) microphone, and the like. The microphone 108 may be positioned at any location on the device 100, including around its periphery, on a top surface, on a back surface, and the like. In some embodiments, the microphone 108 is embedded into a casing of the device 100. In some embodiments, the microphone 108 is detachable from the device 100, for manipulation by the subject.

One or more tasks related to speech may be presented to the subject via the touchscreen display 102 of the device 100. Alternatively or in combination therewith, speech-related tasks may be presented via the microphone 108, which in some embodiments can also act as a speaker. Example tasks include asking the subject to utter certain vowels, produce certain sound sequences for a given duration, and say certain words. Other speech-based tasks may be presented to the subject via the device 100, whereby one or more speech sample is acquired via the microphone 108.

Similarly to the handwriting tasks, one or more external stimuli may be used as a trigger to begin (and in some cases end) each a speech task. In some embodiments, the stimulus for data acquisition using a first modality is the same as that used for data acquisition using a second modality. For example, a sound cue of a same frequency and a same duration is used for both modalities. In this manner, when considering kinematic data obtained from the same subject using the two different modalities, there is no need to account for differences in the trigger used to begin the tasks.

A third modality for acquiring kinematic data using the device 100 relates to natural movement of the subject. Natural movement includes basic locomotion, such as walking, running, climbing or crawling, as well as manipulative movements such as lifting, carrying, throwing and catching. Kinematic data related to natural movement is acquired by the device 100 using an accelerometer 110. The accelerometer 110 measures acceleration, i.e. the rate of change of the velocity of an object. In this case, the object is the device 100, as it is displaced in space (x, y, z) by the subject. A natural movement sample is obtained during a natural movement related task.

Any technology suitable for measuring proper acceleration, by converting mechanical motion into an electrical signal, may be used. For example, the accelerometer 110 may comprise piezoelectric, piezoresistive and/or capacitive components. In some embodiments, the accelerometer 110 is MEMs-based, such as a thermal accelerometer. In some embodiments, the accelerometer 110 and the microphone 108 are provided as a single device, as accelerometers can also be used to record sound. Various embodiments may apply depending on practical implementation. The accelerometer may also incorporate a gyroscope and may be embedded in an Inertial measurement unit (IMU). An IMU is a platform which contains one or more inertial sensors (accelerometer, gyroscope, magnetometer) to assess linear acceleration, angular velocity and magnetic North. IMUs are sometimes referred to as magnetic and inertial measurement unit (MIMU), magnetic angular rate and gravity sensor (MARG), inertial and magnetic measurement unit (IMMU), or attitude and heading reference system (AHRS), depending on configuration and domain of application.

One or more tasks related to natural movement may be presented to the subject via the touchscreen display 102 of the device 100 and/or the microphone 108. An example task includes asking the subject to hold the device 100 still with arms outstretched, for a given duration of time. Another example task includes asking the subject to draw a given shape, such as a triangle or a circle, in the air with his or her arms while holding the device 100, or to draw a shape in the air as large as possible while holding the device 100. Yet another example task includes asking the subject to hold the device 100 at a first position, such as straight above his or her head, and to bring the device to a second position, such as at waist level, in a slow and steady manner. Various other tasks relating to natural movement may be presented, depending on practical implementation.

One or more external stimuli may be used as a trigger to begin (and in some cases end) each natural movement task. The same stimuli may be used to trigger the beginning of a natural movement task as that used for a handwriting task and/or a speech task.

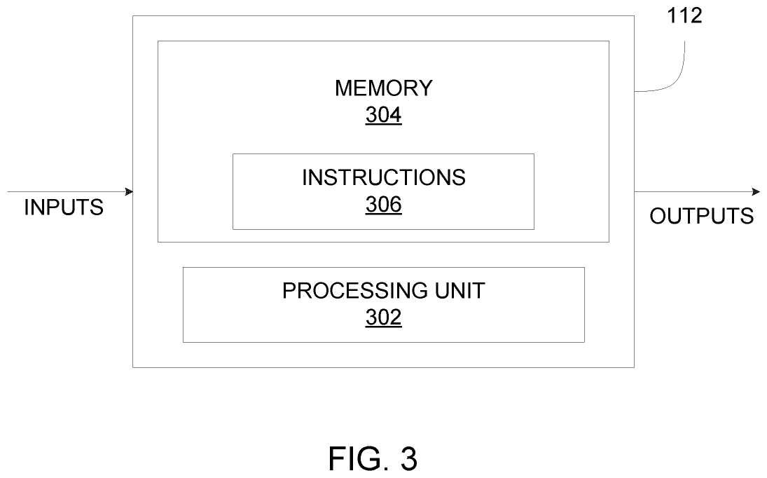

Once the handwriting sample(s), speech sample(s), and/or natural movement sample(s) are acquired by the different modalities of the device 100, they are transmitted to a controller 112 on the device 100. In some embodiments, the controller 112 comprises a processing unit 302 and a memory 304 which has stored therein computer-executable instructions 306, as illustrated in FIG. 3. The processing unit 302 may comprise, for example, any type of general-purpose microprocessor or microcontroller, a digital signal processing (DSP) processor, a CPU, an integrated circuit, a field programmable gate array (FPGA), a reconfigurable processor, a graphics processing unit (GPU), other suitably programmed or programmable logic circuits, or any combination thereof.

The memory 304 may comprise any suitable known or other machine-readable storage medium. The memory 304 may comprise non-transitory computer readable storage medium, for example, but not limited to, an electronic, magnetic, optical, electromagnetic, infrared, or semiconductor system, apparatus, or device, or any suitable combination of the foregoing. The memory 504 may be, for example random-access memory (RAM), read-only memory (ROM), electro-optical memory, magneto-optical memory, erasable programmable read-only memory (EPROM), electrically-erasable programmable read-only memory (EEPROM), Ferroelectric RAM (FRAM) or the like. Memory 304 may comprise any storage means (e.g., devices) suitable for retrievably storing machine-readable instructions 306 executable by processing unit 302.

The program instructions 306 may be implemented in a high level procedural or object oriented programming or scripting language, or a combination thereof. Alternatively, the program instructions 306 may be implemented in assembly or machine language. The language may be a compiled or interpreted language. Program code may be stored on a storage media or a device, for example a ROM, a magnetic disk, an optical disc, a flash drive, or any other suitable storage media or device. The program code may be readable by a general or special-purpose programmable computer for configuring and operating the computer when the storage media or device is read by the computer to perform the procedures described herein.

The computer-executable program instructions 306 may be in many forms, including program modules, executed by one or more computers or other devices. Generally, program modules include routines, programs, objects, components, data structures, etc., that perform particular tasks or implement particular abstract data types. Typically the functionality of the program modules may be combined or distributed as desired in various embodiments.

The program instructions 306 are configured to present the tasks to the subject on the device 100 and to acquire the kinematic data using the different modalities. The program instructions 306 may also be configured to generate the external stimuli to begin (and in some cases end) the tasks, and to coordinate acquisition of the data with the beginning and end of the various tasks.

The program instructions 306 may be configured to present the tasks for the different modalities in a specific order. For example, the subject may be asked to perform handwriting tasks, followed by speech tasks, followed by natural movement tasks; or speech tasks, followed by natural movement tasks, followed by handwriting tasks. The modalities may be alternated, for example: one handwriting task, one speech task, one handwriting task, one natural movement task, one speech task, one natural movement task. The complexity level of the tasks may also be varied, for example by presenting increasingly difficult tasks. Various combinations may be presented to the subject in order to obtain a maximum amount of information that may be used in the motor assessment.

Depending on the task, the kinematic data allows to study different neuromotor functions. For example a task triggered by a visual or an audio stimuli involves the visual or the auditive areas and may be based on the simple reaction test (SRT). It examines the capacity of the subject to react as quickly as possible to a visual or an audio stimulus. Using left or right visual stimuli to determine the direction of a stroke evaluates the capacity of a subject to react quickly and to make a good choice at the same time, both characteristics being of equal importance. Speed accuracy trade-off tasks can assess the ability of the subject to coordinate spatial and temporal properties of his or her movements under competing speed (temporal) and accuracy(spatial) requirements. Other tasks might evaluate the rhythmical properties of the subject as his/her forearm or hand is oscillating at maximum frequency.

The tasks using the different modalities may need to be performed within a given time frame, such as 15 minutes, 30 minutes, or any other suitable time frame. The program instructions 306 may be configured to enforce the time requirements, such as by presenting a timer on the touchscreen display 102, or by triggering a timer on the device 100 separate from the touchscreen display 102.

The program instructions 306 may be configured to generate a file with the handwriting sample(s), speech sample(s), and natural movement sample(s) together with subject data. Subject data may comprise identification data of the subject. In some embodiments, the identification data excludes personal information about the subject (such as name, date of birth, etc.) in order to protect the identity of the subject. For example, a subject number may instead be used. Alternatively, detailed personal information may be stored with the kinematic data. In some embodiments, subject data includes information about the device used to acquire the data, for example a hardware identifier. In some embodiments, subject data comprises information about the data acquisition, such as the time and/or day on which it was acquired, whether it was acquired using the left or right hand, etc. A questionnaire can also be used to evaluate some specific conditions, like sleep time, cardiac frequency, etc., or this information can be provided by any device, such as a cell phone, an instrumented watch, and the like.

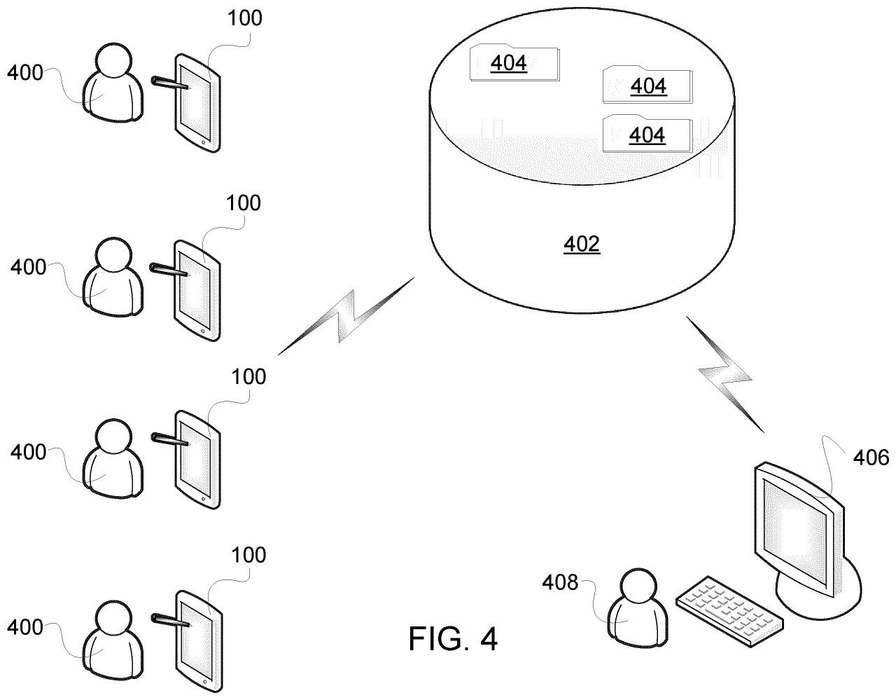

In some embodiments, the file comprising the kinematic data and the subject data is transmitted to a remote location. An example is illustrated in FIG. 4, where a plurality of subjects 400 are each assigned a device 100 for acquisition of kinematic data. The subjects 400 can have their devices 100 with them in their home, or they can access the devices at specific locations such as a health center, a recreational center, a rehab center, and the like. The files 404 are transmitted to a database 402, which may be accessible through a cloud-based service (i.e. Dropbox™, Amazon™ Web Services, Cloud SQL™ etc) or through an internal local area network (LAN) or wide area network (WAN). The files 404 may be accessed from another computing device 406 by an operator 408. In some embodiments, the operator 408 is a clinician or other medical professional involved in the evaluation of neuromotor diseases.

Each file 404 links the data to a user 400 and to a device 100. The data may be processed to provide a neuromotor assessment of the subject based on at least two of the handwriting sample, the speech sample, and the natural movement sample, by comparatively assessing kinematics associated with the samples. In some embodiments, analysis of the data is performed by the controller 112 on the mobile computing device 100. In some embodiments, analysis of the data is performed remotely from the mobile computing device 100, for example on computing device 406. Where the analysis is performed may be determined as a function of the processing capabilities of the mobile computing device 100, and more specifically of the processing unit 302 of the controller 112.

The network setup, as illustrated in FIG. 4, provides the ability of compiling a large amount of information regarding different muscle groups as well as central information about the brain controller, which can be used for research purposes. The setup also facilitates general monitoring and assessment of subjects from health care providers without requiring face-to-face evaluations.

In some embodiments, updates and/or new features for the software residing on the controller 112 of each device 100 may be pushed to the devices 100 through the network, from the computing device 406 or another computing device. The software may therefore be managed remotely.

The kinematic data acquired using the various types of tasks described herein, namely handwriting, speech and natural movement, may be analysed using the Kinematics Theory of rapid human movements (Plamondon, R.: A kinematic theory of rapid human movements. Part I: Movement representation and generation Biological Cybernetics. 72, 295-307 (1995)). Depending on the modality with which the kinematic data is acquired, the data may represent any one of a position in space, velocity, and acceleration. According to the Kinematics Theory, the way in which neuromuscular systems are involved in the production of muscular movements is modelled using lognormal velocity profiles, which may be referred to as the Delta-lognormal or the Sigma-lognormal model.

The handwriting sample corresponds to a plurality of positions in space of the tip of the stylus 106 or a finger of the hand 104, as a function of time. The positions may be derived with respect to time to obtain velocity. The velocity may be derived with respect to time to obtain acceleration. The Kinematic Theory of rapid human movements may be applied to the data obtained from the handwriting samples to extrapolate information about the subject and perform the neuromotor assessment.

The natural movement sample corresponds to an acceleration, which may be integrated over time to obtain velocity. The velocity may be integrated over time to obtain positions in space as a function of time. The Kinematic Theory of rapid human movements may be applied to the data obtained from the natural movement samples to extrapolate information about the subject and perform the motor assessment.

The speech data may be converted to a velocity profile (or a velocity signal). In some embodiments, the conversion is performed as described in Carmona, C., Plamondon, R., Gómez, P., Ferrer, M. A., Alonso, J. B., and Londral, A. R. (2016). “Application of the lognormal model to the vocal tract movement to detect neurological diseases in voice,” in Innovation in Medicine and Healthcare, Smart Innovation, Systems and Technologies, Vol. 60, eds Y. W. Chen, S. Tanaka, R. J. Howlett, and L. C. Jain (Cham: Springer International Publishing AG), 25-35. The velocity profile may be integrated over time to obtain positions in space as a function of time. The velocity profile may be derived with respect to time to obtain acceleration. The Kinematic Theory of rapid human movements may be applied to the data obtained from the speech samples to extrapolate information about the subject and perform the motor assessment.

The ability to obtain kinematic data from a subject, using different modalities, on a single device, allows comparative analyses to be performed that can draw parallels between information obtained from the different modalities. New information may be derived that would not otherwise be available. Neuromotor assessments of a subject can be performed in a more thorough and complete manner, providing a global perspective of the subject's abilities both at the central and the peripheral level of his neuromotor control, based on information acquired in a synchronized and controlled environment.

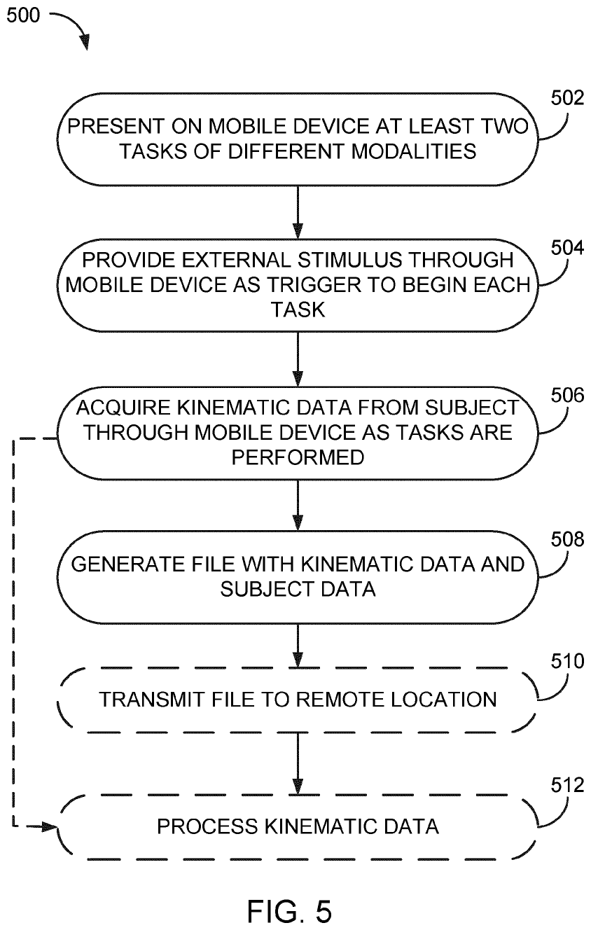

Referring to FIG. 5, there is illustrated a method 500 of obtaining kinematic data from a subject, for neuromotor assessment of the subject. At step 502, at least two tasks of different modalities are presented on a mobile computing device, such as device 100. Any two tasks from a handwriting task, a speech task, and a natural movement task may be presented, each task executable by the subject with the mobile computing device.

At step 504, an external stimulus is provided through the mobile computing device as a trigger to begin each task. Although presented sequentially, it will be understood that steps 502 and 504 are performed iteratively, for example a first task is presented with a first external stimulus, followed by a second task with a second external stimulus, etc. As indicated above, the tasks may be presented in a specific and predetermined order and/or manner.

At step 506, kinematic data is acquired from the subject on the mobile computing device as the tasks are being performed. For example, kinematic data is acquired from the touchscreen display 102 during handwriting tasks, from the microphone 108 during speech tasks, and from the accelerometer 110 during natural movement tasks.

At 508, a file is generated with the kinematic data together with subject data. In some embodiments, the file is transmitted to a remote location at step 510 for further processing. In some embodiments, the method 500 comprises a step 512 of processing the kinematic data to get parameters that characterize a neuromotor performance of the subject. The processing of step 512 is performed remotely when step 510 is also performed, and is performed locally when step 510 is omitted. In some embodiments, the processing step 512 is performed locally and the data is also transmitted to a remote location at step 510. In some embodiments step 508 is omitted and the processing step 512 is performed locally.

The kinematic data may be processed in a same manner independently of a source of the kinematic data, such that data obtained from the handwriting task, the speech tasks, and the natural movement task is treated in a coherent and uniform manner. In some embodiments, processing the kinematic data comprises decomposing complex movements represented by the kinematic data into simple movements, for example by applying the Kinematics Theory of rapid human movements. This may be done using Sigma-lognormals or other models having similar characteristics. Other embodiments may also apply for decomposing the complex movements into simple movements. Simple movements may be analyzed statically and a neuromotor performance of the subject may be characterized based on the analysis.

Using the method 500, both the central controller and the peripheral executor of the brain may be studied simultaneously through the global perspective afforded from the acquisition of kinematic data using two or more modalities. Neuromotricity of subjects may thus be assessed optimally.

The above description is meant to be exemplary only, and one skilled in the art will recognize that changes may be made to the embodiments described without departing from the scope of the invention disclosed. For example, in some embodiments, the different tasks may be integrated or hidden, such as in a video game environment, to motivate subjects to use the system, particularly children, and to facilitate large data collection across large populations, opening the door to data mining and deep learning neuromotor assessments.

Still other modifications which fall within the scope of the present invention will be apparent to those skilled in the art, in light of a review of this disclosure. For example, although illustrated as a local application that resides on the mobile computing device 100, the program instructions 306 may reside in whole or in part remotely from the device 100. In some embodiments, an application is downloaded onto the device 100. In some embodiments, the features described herein are provided through the device 100 on a web-based platform.

Various aspects of the methods and devices for neuromotor assessment may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments. The scope of the following claims should not be limited by the embodiments set forth in the examples, but should be given the broadest reasonable interpretation consistent with the description as a whole.

1. A method for obtaining kinematic data from a subject, the method comprising:
presenting on a mobile computing device at least two tasks from: a handwriting task, a speech task, and a natural movement task, the at least two tasks executable by the subject with the mobile computing device;
providing an external stimulus through the mobile computing device as a trigger to begin the at least two tasks;
acquiring kinematic data from the subject on the mobile computing device as the at least two tasks are being performed; and
generating a file with the kinematic data together with subject data.

2. The method of claim 1, further comprising processing the kinematic data to get parameters that characterize a neuromotor performance of the subject.

3. The method of claim 2, wherein processing the kinematic data comprises processing the kinematic data on the mobile computing device.

4. The method of claim 2, wherein processing the kinematic data comprises comparing and correlating data from different neuromotor groups acquired through the at least two tasks.

5. The method of claim 2, wherein processing the kinematic data comprises:
decomposing complex movements into simple movements;
analyzing statically the simple movements; and
characterizing the neuromotor performance of the subject based on the analyzing.

6. The method of claim 1, wherein providing the external stimulus comprises providing a same external stimulus for the at least two tasks.

7. The method of claim 1, wherein providing the external stimulus comprises signaling an end to the at least two tasks.

8. The method of claim 1, further comprising transmitting the file with the kinematic data to a remote location.

9. The method of claim 1, wherein the subject data comprises a hardware identifier for the mobile computing device.

10. The method of claim 1, wherein the tasks are presented on the mobile device in accordance with at least one of a specific order and a complexity level.

11. A system for obtaining kinematic data from a subject, the system comprising:
a processing unit; and
a non-transitory computer readable medium having stored thereon program code executable by the processing unit for:
presenting on a mobile computing device at least two tasks from: a handwriting task, a speech task, and a natural movement task, the at least two tasks executable by the subject with the mobile computing device;
providing an external stimulus through the mobile computing device as a trigger to begin the at least two tasks;
acquiring kinematic data from the subject on the mobile computing device as the at least two tasks are being performed; and
generating a file with the kinematic data together with subject data.

12. The system of claim 11, wherein the program code is further executable for processing the kinematic data to get parameters that characterize a neuromotor performance of the subject.

13. The system of claim 12, wherein processing the kinematic data comprises processing the kinematic data on the mobile computing device.

14. The system of claim 12, wherein processing the kinematic data comprises comparing and correlating data from different neuromotor groups acquired through the at least two tasks.

15. The system of claim 12, wherein processing the kinematic data comprises:
decomposing complex movements into simple movements;
analyzing statically the simple movements; and
characterizing the neuromotor performance of the subject based on the analyzing.

16. The system of claim 11, wherein providing the external stimulus comprises providing a same external stimulus for the at least two tasks.

17. The system of claim 11, wherein providing the external stimulus comprises signaling an end to the at least two tasks.

18. The system of claim 11, wherein the program code is further executable for transmitting the file with the kinematic data to a remote location.

19. The system of claim 11, wherein the subject data comprises a hardware identifier for the mobile computing device.

20. The system of claim 11, wherein the tasks are presented on the mobile device in accordance with at least one of a specific order and a complexity level.
